(19) United States
(12) Patent Application Publication
(10) Pub. No.: US 2011/0160823 A1
(43) Pub. Date: Jun. 30, 2011

(54) IMPLANTABLE LEADS WITH A LOW COEFFICIENT OF FRICTION MATERIAL

(76) Inventors: Andrew De Kock, Andover, MN (US); Kimberly A. Morris, Minneapolis, MN (US); Ronald W. Kunkel, Jim Falls, WI (US); Peter J. Wolf, Dresser, WI (US)

(21) Appl. No.: 12/959,010

(22) Filed: Dec. 2, 2010

Related U.S. Application Data
(60) Provisional application No. 61/291,097, filed on Dec. 30, 2009.

(51) Int. Cl. A61N 1/05; B29C 33/42; C23F 1/04

(52) U.S. Cl. 607/116; 264/219; 216/83

(57) ABSTRACT

A method of reducing a coefficient of friction between a medical electrical lead and a delivery system includes machining a mold for a lead, roughening at least a portion of the mold to an average surface roughness of at least about 7 micro-inches and injecting a polymer into the roughened mold to form a roughened portion of a lead body.

### CROSS REFERENCE TO RELATED APPLICATIONS

This application claims the benefit under 35 U.S.C. §119 of U.S. Provisional Application No. 61/291,097, filed on Dec. 30, 2009, entitled “Implantable Leads with a Low Coefficient of Friction Material,” which is incorporated herein by reference in its entirety for all purposes.

### TECHNICAL FIELD

The present invention relates to methods of inserting a medical electrical lead through a delivery system for use in cardiac rhythm management systems. In particular, the present invention relates to methods of reducing the amount of force necessary to insert a medical electrical lead through a delivery system.

### BACKGROUND

Cardiac pacemaker leads are generally designed to be delivered into a patient's body through the use of a delivery system, such as a catheter or sheath. As the lead is inserted through the catheter, frictional forces between the lead and the catheter may restrict the ease with which the lead is advanced through the catheter. If the frictional forces are too high, the lead body can begin to buckle proximal to the delivery system tools. Lowering the frictional forces between the lead and the catheter can prevent buckling or kinking of the lead body as the lead is advanced through the catheter.

One method of reducing the frictional forces between the lead and the catheter is to lower the coefficient of friction between the outer surface of the lead and the inner surface of the catheter. As the coefficient of friction is reduced, the force required to insert the lead into and/or withdraw the lead from, the catheter is also reduced.

### SUMMARY

Discussed herein are various methods for reducing the coefficient of friction between an implantable medical electrical lead and a delivery system, such as a catheter.

Example 1 is a method of forming a lead body for a medical electrical lead having a roughened outer surface including the steps of: roughening at least a portion of the inner surface of a mold cavity to an average surface roughness (Ra) of at least 7 micro-inches; injecting a polymeric material into the roughened cavity for forming the lead body; and curing the polymeric material to form a lead body having an outer surface wherein at least a portion of the outer surface is roughened to an average surface roughness (Ra) of at least about 7 micro-inches.

In Example 2, wherein a maximum peak to valley distance is less than about 800 micro-inches.

In Example 3 the method according to any one of Examples 1 or 2, further including roughening the inner surface of the cavity to an average surface roughness ranging from about 25 micro-inches to about 90 micro-inches and having maximum peak to valley distance of less than about 800 micro-inches.

In Example 4, the method according to any one of Examples 1-3, further including roughening the inner surface of the cavity to an average surface roughness ranging from about 7 micro-inches to about 65 micro-inches and having maximum peak to valley distance of less than about 800 micro-inches.

In Example 5, the method according to any one of Examples 1-4, further including roughening the inner surface of the cavity to an average surface roughness ranging from about 7 micro-inches to about 65 micro-inches and having a maximum peak to valley distance of less than about 650 micro-inches.

In Example 6, the method according to any one of Examples 1-5, further including roughening the inner surface of the cavity to an average surface roughness ranging from about 40 micro-inches to about 65 micro-inches and having a maximum peak to valley distance of less than about 650 micro-inches.

In Example 7, the method according to any one of Examples 1-6, wherein the polymeric material comprises silicone.

In Example 8, the method according to any one of Examples 1-7, wherein roughening at least a portion of the mold comprises any one of bead blasting the mold, wire electric discharge machine burnishing the mold or chemical etching the mold.

In Example 9, the method according to any one of Examples 1-8, wherein silicone rubber is injected into the mold cavity to form a distal tip region of the lead body.

Example 10 is a method of roughening an outer surface of at least a portion of a lead body for a medical electrical lead including the steps of machining a mold including a cavity for forming a lead body such that an inner surface of the mold cavity is substantially smooth; wire electric discharge machine burnishing the inner surface of the mold cavity to roughen at least a portion of the inner surface of the cavity to an average surface roughness (Ra) ranging from about 7 micro-inches to about 90 micro-inches and having a maximum peak to valley distance of less than about 800 micro-inches; injecting a polymeric material comprising silicone into the roughened cavity for forming the lead body; and curing the polymeric material to form a lead body having an outer surface wherein at least a portion of the outer surface is roughened to an average surface roughness (Ra) ranging from about 7 micro-inches to about 90 micro-inches and having a maximum peak to valley distance of less than about 800 micro-inches.

In Example 11, the method according to Example 10, further including roughening the inner surface of the cavity to an average surface roughness ranging from about 25 micro-inches to about 90 micro-inches.

In Example 12, the method according to any one of Examples 10-11, further including roughening the inner surface of the cavity to an average surface roughness ranging from about 7 micro-inches to about 65 micro-inches and having a maximum peak to valley distance of less than about 650 micro-inches.

In Example 13, the method according to any one of Examples 10-12, further including comprising roughening the inner surface of the cavity to an average surface roughness ranging from about 40 micro-inches to about 65 micro-inches and having a maximum peak to valley distance of less than about 650 micro-inches.

In Example 14, the method according to any one of Examples 10-13, wherein silicone rubber is injected into the cavity to form a distal tip region of the lead body.

Example 15 is a medical electrical lead having a reduced contact area including: an elongated polymeric lead body extending from a proximal end adapted to be coupled to a pulse generator to a distal end, wherein at least a portion of the elongated polymeric lead body has a roughened outer surface having an average surface roughness ranging from about 25 micro-inches to about 90 micro-inches and a maximum peak to valley distance less than about 800 micro-inches; at least one conductor extending within the lead body from the proximal end in a direction towards the distal end; and at least one electrode located on the lead body and operatively coupled to the at least one conductor.

In Example 16, the medical electrical lead according to Example 15, wherein the roughened outer surface has an average surface roughness ranging from about 40 micro-inches to about 90 micro-inches.

In Example 17, the medical electrical lead according to any one of Examples 15 or 16, wherein the roughened outer surface has an average surface roughness ranging from about 7 micro-inches to about 65 micro-inches and a maximum peak to valley distance of less than about 650 micro-inches.

In Example 18, the medical electrical lead according to any one of Examples 15-17, wherein the roughened outer surface has an average surface roughness ranging from about 40 micro-inches to about 65 micro-inches and a maximum peak to valley distance of less than about 650 micro-inches.

In Example 19, the medical electrical lead according to any one of Examples 15-18, wherein the at least one portion of the lead body having the roughened outer surface comprises silicone.

In Example 20, the medical electrical lead according to any one of claims 15-19, wherein the at least one portion is a distal tip region of the lead body, wherein the distal tip region comprises silicone.

In Example 21, the medical electrical lead according to any one of claims 15-20, wherein the lead body further comprises two or more portions having a roughened outer surface, wherein the two or more portions of the lead body having the roughened outer surface comprise silicone.

Example 22 is a method of forming a lead body for a medical electrical lead having a roughened outer surface including the steps of: roughening at least a portion of the inner surface of the cavity to an average surface roughness (Ra) of at least 7 micro-inches and having a maximum peak to valley distance of less than about 800 micro-inches; and injecting a polymeric material into the roughened cavity for forming the lead body; and curing the polymeric material to form a lead body having an outer surface wherein at least a portion of the outer surface is roughened to an average surface roughness Ra of at least 7 micro-inches and having a maximum peak to valley distance of less than about 800 micro-inches.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a perspective view of a medical electrical lead according to one embodiment of the present invention.

FIG. 2 is a perspective view of a medical electrical lead according to another embodiment of the present invention.

FIG. 3 is a schematic, partial cross-sectional view of a portion of a lead disposed within a portion of a delivery catheter according to an embodiment of the present invention.

FIGS. 4A and 4B are schematic views of a lead body including at least one portion having a roughened outer surface in accordance with various embodiments of the present invention.

FIG. 5 is a flow chart of an exemplary method of manufacturing a lead body having a roughened outer surface in accordance with various embodiments of the present invention.

FIG. 6 is a scatter plot of the maximum insertion force versus the average surface roughness (Ra) for a set of sample lead bodies provided in accordance with the various embodiments of the present invention.

FIG. 7 is a plot of the maximum peak to valley distance (PV) versus the average surface roughness (Ra) for a given set of sample lead bodies provided in accordance with the various embodiments of the present invention.

FIG. 8 is a graph showing the linear relationship between surface roughness and the maximum peak to valley distance (PV) at the 95% UCI for a set of sample lead bodies provided in accordance with the present invention.

FIG. 9 is a graph showing the relationship between average surface roughness (Ra) and the maximum insertion force for a given set of sample lead bodies calculated at the 95% UCI for both insertion force and peak to valley distance (PV).

FIG. 10 is a graph showing the relationship between catheter clearance and maximum insertion force at a constant average surface roughness for a given set of sample lead bodies.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

Medical electric leads are generally introduced into a patient's body using a delivery system, such as a catheter. The present invention provides methods of reducing the coefficient of friction between an outer surface of a lead body and an inner surface of a delivery system, such as a catheter. Leads generally include a flexible tubular lead body having a proximal end, a distal end and an outer surface along a length between the proximal and distal ends. As the lead body is advanced through the catheter and the outer surface of the lead body contacts an inner surface of the catheter, the contact may cause friction and resistance to movement between the two surfaces.

The leads according to the various embodiments of the present invention are suitable for sensing intrinsic electrical activity and/or applying therapeutic electrical stimuli to a patient. Exemplary applications include, without limitation, cardiac rhythm management (CRM) systems and neurostimulation systems. For example, in exemplary CRM systems utilizing pacemakers, implantable cardiac defibrillators, and/or cardiac resynchronization therapy (CRT) devices, the medical electrical leads according to the various embodiments of the invention can be endocardial leads configured to be partially implanted within one or more chambers of the heart so as to sense electrical activity of the heart and apply a therapeutic electrical stimulus to the cardiac tissue within the heart. Additionally, the leads formed according to the various embodiments of the present invention may be suitable for placement in a coronary vein adjacent to the left side of the heart so as to facilitate bi-ventricular pacing in a CRT or CRT-D system. Still additionally, leads formed according to embodiments of the present invention may be configured to be delivered intravascularly to deliver an electrical stimulation therapy to a nerve or other neurostimulation target. The medical electrical leads may be unipolar, bipolar, or multi-polar depending upon the type of therapy to be delivered.

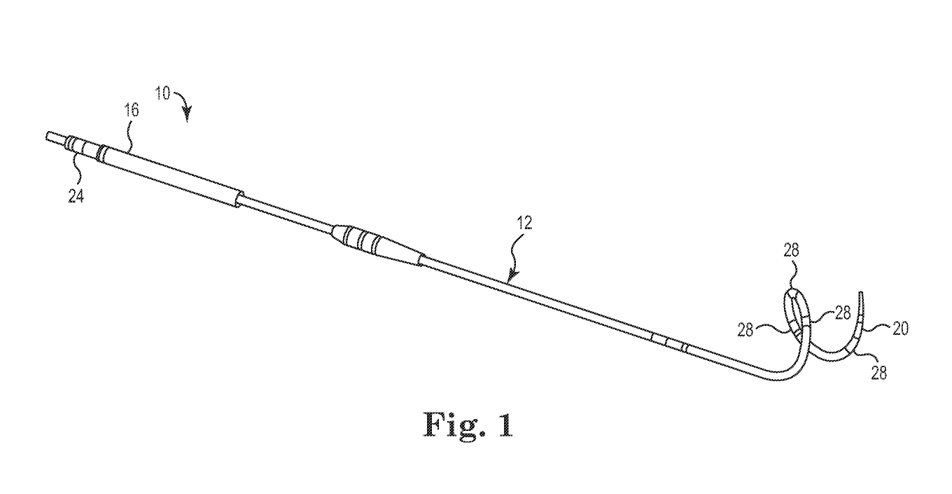

FIG. 1 is a perspective view of a medical electrical lead 10 according to various embodiments of the present invention. According to some embodiments, the medical electrical lead 10 can be configured for implantation within a patient's heart or within a patient's neurovascular regions. The medical electrical lead 10 includes an elongated, polymeric lead body 12 extending from a proximal end 16 to a distal end 20. In one embodiment, the distal end 20 has a tapered profile. The proximal end 16 of the lead body 12 is configured to be operatively connected to a pulse generator via a connector 24. At least one conductor (not shown) extends from the connector 24 through the lead body 12 to one or more electrodes 28 at the distal end 20 of the lead 10. The conductor can include coiled conductors, cable conductors or combinations thereof. In one embodiment, the lead 10 is a quadri-polar lead including one coiled conductor and three cable conductors. The coiled conductors can have either a co-radial or a co-axial configuration. In embodiments of the present invention employing multiple electrodes 28 and multiple conductors, each conductor is connected to an individual electrode 28 in a one-to-one manner allowing each electrode 28 to be individually addressable.

The electrodes 28 can have any electrode configuration as is known in the art. According to one embodiment of the present invention, at least one electrode can be a ring or partial ring electrode. According to another embodiment, at least one electrode 28 is a shocking coil. In some embodiments, a combination of electrode configurations may be used. The electrodes 28 can be coated with or formed from platinum, stainless steel, MP35N, a platinum-iridium alloy, or another similar conductive material. In further embodiments, a steroid eluting collar may be located adjacent to at least one electrode 28.

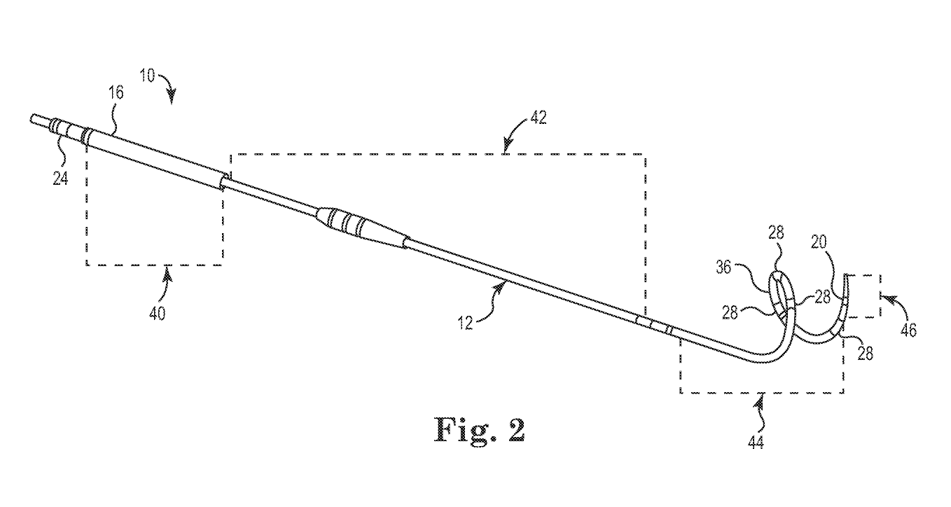

According to various embodiments, the lead body 12 can include one or more fixation members for securing and stabilizing the lead body 12 including the one or more electrodes 28 at a target site within a patient's body. The fixation member(s) can be active or passive. Examples of passive fixation include pre-formed distal portions of the lead body 12 such as, for example, a spiral 36 (FIG. 2), adapted to bear against the vessel walls and/or expandable tines provided at the distal end of the lead body 12. In some embodiments, the fixation member can be a screw-in fixation member. In other embodiments, the fixation member can be an extendable/retractable fixation member and can include one or more mechanical components adapted to facilitate the extension/retraction of the fixation member. An exemplary extendable/retractable fixation member is shown and described in U.S. Pat. No. 6,444,334 which is herein incorporated by reference.

The lead body 12 is flexible, but substantially non-compressible along its length and, in some embodiments, has a circular cross-section. Other lead body cross-sections can also be employed. According to one embodiment of the present invention, an outer diameter of the lead body 12 ranges from about 2 to about 15 French. Additionally, the lead body 12 can be a multi-lumen lead body including at least two lumens. The lumens can have a variety of cross-sectional shapes and can be of the same or different sizes. The lumens facilitate passage of the conductor from the connector 24 to the electrode and/or can receive a guiding element such as a guidewire or a stylet for delivery of the lead 10 to implant the lead 10 within a patient's heart.

The polymeric material used to form the lead body 12 can include a variety of different biocompatible polymeric materials, polymeric material blends, co-block polymers, co-polymers and elastomers used to manufacture lead bodies known to those of skill in the art. Exemplary polymeric materials include, but are not limited to, silicone, polyurethane, polyethylene teraphthalate, polytetrafluoroethlyene and fluorinated ethylene propylene. Other exemplary materials suitable for use as lead body materials include, but are not limited to block co-polymer elastomers, polyurethane, polyurethane blends, polyurethane co-polymers, silicone rubbers, styrene-isobutylene-styrene (SIBS) co-polymers and the like. In one embodiment at least a portion of the lead body 12 is composed of silicone rubber.

According to various embodiments of the present invention, as shown in FIG. 2, the lead body 12 is a multi-lumen lead body 12 and includes at least two portions, the approximate boundaries of which are illustrated by dashed lines. The portions of the lead body 12 can include a proximal portion 40, a middle portion 42, and a distal portion 44 including a lead tip portion 46. The proximal portion 40 generally represents portions of the lead body 12 that connect to the PG and/or lie subcutaneously in the patient's body. The middle portion 42 generally represents portions of the multi-lumen lead body 12 that reside in vessels that lead to the heart and/or in the upper chambers of the heart such as, for example, the right atrium. The distal portion 46 generally represents portions of the lead body 12 that reside within the heart, and generally includes at least one of the electrodes 28. In one embodiment, as shown in FIG. 2, the distal portion 46 also includes the pre-formed spiral fixation member 36. The lead tip portion 46 generally represents the distal end 20 of the lead body 12. The portions illustrated in FIG. 2 can vary in length and/or position on the multi-lumen lead body 12 depending on the type and size of the lead 10, the intended treatment and/or the intended implantation procedure.

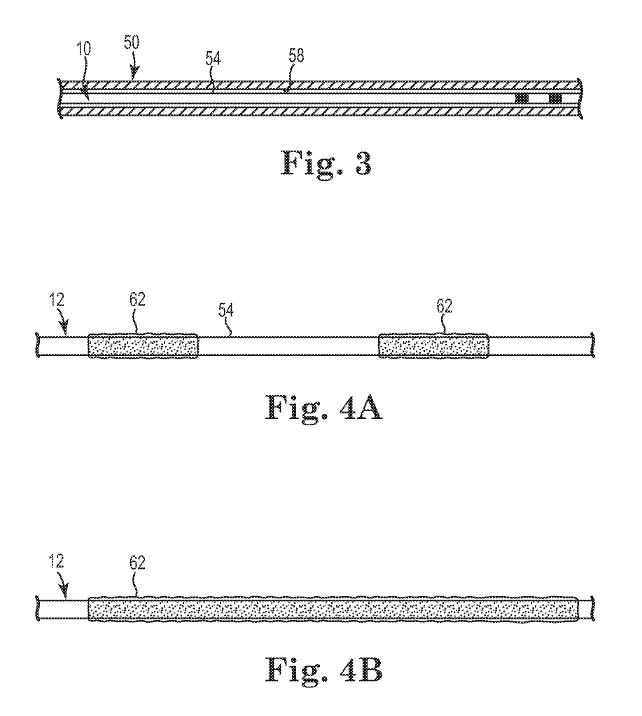

The lead 10 may be delivered to a number of sites within a patient's body using a variety of delivery tools and/or methods. Exemplary delivery tools suitable for delivering the lead 10 to its desired location include guidewires, finishing wires, stylets, delivery catheters and/or combinations thereof. In one embodiment, the lead 10 is advanced to a target location within a patient's body using a delivery catheter. FIG. 3 shows a portion of a lead 10 disposed within a portion of an exemplary delivery catheter 50.

In an exemplary embodiment, the coefficient of friction between an outer surface 54 of the lead body 12 and an inner surface 58 of a delivery system (e.g. catheter 50) is lowered by reducing the contact area of a portion of the lead body 12 that comes into contact with the delivery system. One method of reducing the contact area between the delivery system and the lead body 12 is to increase the surface roughness of at least one portion of the lead body 12.

FIGS. 4A and 4B are schematic views of a lead body 12 including at least one portion 62 having a roughened outer surface 54. In one embodiment, an entire length of the lead body 12 extending from a proximal end 16 to a distal end 20 includes a roughened outer surface. In another embodiment, only the distal portion 44 of the lead body 12 (FIG. 2) includes a roughened outer surface 54. In still other embodiments, only those portions in which the outer surface 54 of the lead body 12 includes silicone can have a roughened outer surface.

According to some embodiments, the average surface roughness (Ra) of the selected portion or portions 62 of the outer surface 54 of the lead body 12 can be controlled such that the overall insertion force is minimized. In one embodiment, the average surface roughness (Ra) of the selected portion or portions 62 is controlled such that the amount of insertion force does not exceed about 380 grams-force. In another embodiment, the average surface roughness (Ra) is controlled such that the amount of insertion force does not exceed about 250 grams-force.

The average surface roughness (Ra) is the average of a set of individual measurements of a surface's peaks and valleys over a selected length of a given sample such as, for example, a lead body. In general, as the average surface roughness (Ra) of the lead body 12 increases, the insertion force decreases. Additionally, as the average surface roughness (Ra) of the lead body 12 increases, the maximum peak to valley (PV) distance on the roughened surface 54 of the lead body 12 also generally increases. The peak to valley distance or PV is defined as the height distance between the top of the highest peak and the bottom of the lowest valley over a selected length of a given sample such as, for example, a lead body. PV is sometimes referred to as Rmax or Rt. The peak to valley distance (PV) also can affect the dimensional tolerance requirements for the lead body itself and thus, often serves as a limiting factor of the average surface roughness (Ra). Both the average surface roughness (Ra) and the peak to valley distance (PV) can be controlled to maintain a safe level of operation of lead performance and to maintain the insertion force within a desired limit.

In some embodiments, the average surface roughness (Ra) of the portion or portions 62 of the lead body 12 ranges from about 7 micro-inches to about 90 micro-inches and more particularly, from about 25 micro-inches to about 90 micro-inches. In other embodiments, the average surface roughness (Ra) of the portion or portions 62 of the lead body 12 ranges from about 7 micro-inches to about 65 micro-inches and more particularly, from about 40 micro-inches to about 65 micro-inches.

In some embodiments, the maximum peak to valley distance (PV) is 800 micro-inches. In other embodiments, the maximum peak to valley distance (PV) is less than about 800 micro-inches and more particularly, is less than about 650 micro-inches. In still other embodiments, the maximum peak to valley distance (PV) ranges from about 650 micro-inches to about 800 micro-inches.

While the maximum insertion force, average surface roughness (Ra) and peak to valley distance (PV) can be looked at independently from one another, their interdependence upon one another cannot be ignored. In general, the surface roughness (Ra) of a lead body can be determined from the maximum allowable insertion force, provided that the maximum peak to valley distance is maintained within acceptable tolerance limits. In some embodiments, for example, to maintain a maximum insertion force of less than 380 grams-force and a maximum peak to valley distance of less than 800 micro-inches, the average surface roughness (Ra) of a portion or portions 62 of a lead body 12 ranges from about 7 micro-inches to about 90 micro-inches. In other embodiments, to maintain a maximum insertion force of less than 250 grams-force and a maximum peak to valley distance of less than 800 micro-inches, the average surface roughness (Ra) of a portion or portions 62 of a lead body 12 ranges from about 25 micro-inches to about 90 micro-inches. In still other examples, to maintain a maximum insertion force of less than 380 grams-force and a maximum peak to valley distance of less than 650 micro-inches, the average surface roughness (Ra) of a portion or portions 62 of a lead body 12 ranges from about 7 micro-inches to about 65 micro-inches. In still yet other embodiments, to maintain a maximum insertion force of less than 250 grams-force and a maximum peak to valley distance of less than 650 micro-inches, the average surface roughness (Ra) of a portion or portions 62 of a lead body 12 ranges from about 40 micro-inches to about 65 micro-inches.

In addition to roughening the outer surface 54 of a select portion or portions 62 of the lead body 12, the amount of clearance between the outer surface 54 of the lead body 12 and the inner surface 58 of the delivery catheter 50 also can affect the insertion force. Clearance can be defined as the percent difference between the inner diameter of the delivery catheter 50 and the outer diameter of the lead body 12, including the roughened outer surface 54. In one embodiment, the amount of clearance between the delivery catheter 50 and the lead body 12 disposed in the delivery catheter is at least about 6% and more particularly, at least about 10.5%. In other embodiments, the amount of clearance is at least about 14.5%. In still yet other embodiments, the amount of clearance between the catheter 50 and the lead body 12 disposed within the catheter 50 ranges from about 6% to about 18%.

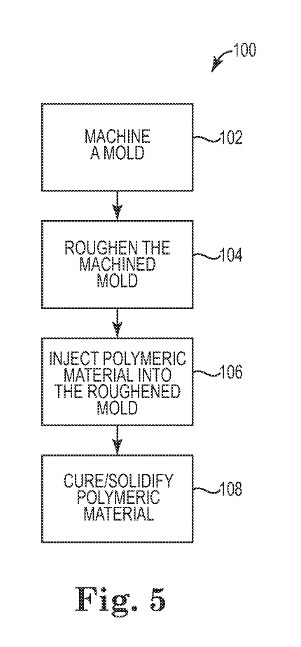

FIG. 5 is a flow chart outlining a method 100 of increasing the surface roughness of a lead body according to an embodiment of the present invention. First, the inner surface of cavity of a mold for forming a lead body is machined to have a substantially smooth surface (Box 102). The inner surface of the cavity is then roughened to increase the surface area of the mold cavity (Box 104). The surface area of the mold cavity is increased by an amount sufficient to reduce the coefficient of friction and the contact area between the lead body and the delivery system.

The machined mold may be roughened by means known in the art. For example, the machined mold can be roughened by bead blasting or wire electric discharge machine (EDM) burnishing. EDM is sometimes referred to as “spark machining” because it removes metal by producing a rapid series of repetitive electrical discharges. These electrical discharges are passed between an electrode and the piece of metal (e.g. the mold) being machined. The small amount of material that is removed from the mold is flushed away with a continuously flowing fluid. The repetitive discharges create a set of successively deeper craters in the work piece until the final shape is produced.

According to one embodiment of the method, the mold cavity is first machined close to the final desired geometry in the steel. Next, a graphite electrode is machined with the reverse of the final cavity configuration but to the final dimensions. The machined block is then placed in a conductive fluid bath on a 3 axis table position directly below the conducting machine head. The graphite electrode is then fixed in position on the electrical conducting head. The graphite electrode is lowered via a CNC program into the machined mold block while electrical charges travel through the graphite electrode and bridge a small gap through the fluid to the mold cavity needing to be roughened. The roughness of a mold is determined by the electrical setting of the EDM machine, the length or electrical arc from the electrode to the mold cavity, the speed of the electrode into the cavity and the electrode material and the surface finish of the electrode. Typically the electrodes are manufactured using a high speed machining center so the surface finish is in the 5-8 Ra range. The major influences of surface roughness in the cavity during the EDM process is the power setting of the EDM machine, the speed of the electrode into the mold cavity and the arc length of the electricity from the electrode to the mold cavity. The process is continued until the final geometry and surface roughness is obtained.

In other embodiments, the machined mold can be roughened by chemical etching the surface of the machined mold using chemical etching techniques well known to those of skill in the art.

In still other embodiments, the mold may be formed from a polymeric material in which case, using an EDM or chemical etching method to roughen the inner surface of the mold cavity is no longer appropriate. In embodiments where a polymeric mold is employed, the inner surface of the mold cavity can be roughened by bead blasting or scraping, sanding or otherwise roughing the inner surface. In a further embodiment, a polymeric mold may be itself molded to include a roughened inner surface.

After the mold has been roughened, a polymeric material that forms the lead body is injected into the roughened mold cavity (Box 106). Examples of suitable polymeric materials include, but are not limited to: silicones, polyurethanes, polyethylene terephthalate (PET), polytetrafluoroethylene (PTFE) and fluorinated ethylene propylene (FEP). An example of a suitable silicone is liquid silicone rubber (LSR). Examples of suitable commercially available liquid silicone rubbers include, but are not limited to: MED-4860, available from Nusil Technology, Carpinteria, Calif. and SILASTIC BioMedical Grade Liquid Silicone Rubber Q7-4860, available from Dow Corning Corporation, Midland Mich.

Once the polymeric material is injected into the roughened mold, the material is allowed to cure or solidify in the mold (Box 108). Because the polymeric material takes the shape of the mold, the polymeric material will also take on the roughness that was created during the molding process. The final lead body is formed once the polymeric material has solidified. In one embodiment, the average surface roughness of one or more regions of the lead body is formed during the molding.

The portion of the lead body 12 with the roughened outer surface 54 facilitates the lead to be inserted and withdrawn from a delivery system using a decreased amount of force. In one embodiment, the force required to insert and/or withdraw a lead body having a roughened surface is less than about 380 grams-force (gf), more particularly, is less than about 250 grams-force, and most particularly, is less than about 200 grams-force. For example, using the contact area reducing method of the present invention, a lead body having an outer diameter of up to about 0.075 inches is compatible with a delivery system having an inner diameter of about 0.087 inches using direct delivery. Direct delivery is a lead delivery technique in which a catheter is used to deliver a lead directly into a branch vein. The catheter tip is delivered through the coronary sinus and bent into a branch vein extending off of the great cardiac vein.

In another embodiment, the coefficient of friction between the lead body and the delivery system can be lowered by either using a lubricious material to form at least part of the lead body or by coating an outer surface of the lead body with a lubricious coating. In an exemplary embodiment, the lubricious material and/or coating is liquid silicone rubber (LSR).

When a lubricious material is used to form at least part of the lead body, the lubricious material can be used as a base for either a pre-molded lead body or an extruded lead body. The lubricious material may also be over-molded or bonded in place to function as the outer surface of the lead body. An example of a suitable commercially available liquid silicone rubber for use as part of a lead body includes, but is not limited to, MED1-4855, available from Nusil Technology, Carpinteria, Calif. Although the lubricious material is discussed as forming part of the lead body, the lubricious material may instead be used to form at least a part of the delivery system without departing from the intended scope of the present invention.

When a lubricious coating is applied onto the lead body, the lubricious coating is applied as a surface coating over an existing substrate material, such as silicone, polyurethane or a rough metallic component. An example of a suitable commercially available liquid silicone rubber for use as a surface coating includes, but is not limited to, LSR1-9716-30 available from Nusil Silicone Technology LCC, Carpinteria, Calif. Although the lubricious coating is discussed as being applied onto the outer surface of the lead body, the lubricious coating may instead be applied to the delivery system without departing from the intended scope of the present invention. For example, the lubricious material may be used to form part of the delivery system or the lubricious coating may be deposited onto an inner diameter of the delivery system.

The present invention is more particularly described in the following examples that are intended as illustrations only, since numerous modifications and variations within the scope of the present invention will be apparent to those skilled in the art. Unless otherwise noted, all parts, percentages, and ratios reported in the following examples are on a weight basis, and all reagents used in the examples were obtained, or are available, from the chemical suppliers described below, or may be synthesized by conventional techniques.

### EXAMPLES

### Equipment Used

Lead: polyurethane/silicone sub-assemblies having a 4 inch distal silicone tip with an approximate outer diameter of 0.071 inches.

Delivery System: RAPIDO Advance Model 7712, an 8 French (Fr) catheter having an inner diameter of 0.087±0.001 inches.

Guide Wire: Whisper View EDS Model 4638.

### Materials Used

SILASTIC BioMedical Grade Liquid Silicone Rubber Q7-4860: a liquid silicone rubber available from Dow Corning Corporation, Midland Mich.

MED 1-4855: a silicone material available from Nusil Silicone Technology LCC, Carpinteria, Calif.

LSR1-9716-30: a low coefficient of friction silicone coating available from Nusil Silicone Technology LCC, Carpinteria, Calif.

### Procedure

Lead Insertion/Withdrawal through an 8 Fr Catheter in Direct Delivery Test

To determine the compatibility between various leads and a RAPIDO Advance catheter, the forces between each of the leads and the catheter were measured as each lead was pushed over a guide wire. The forces are representative of the forces a physician would experience upon introducing the lead through the 8Fr catheter placed in a worst case direct delivery position during delivery of the lead into a body. The clearance between the sample leads and the catheter was about 18%.

The forces were measured on an Interventional Device Test Equipment (IDTE) 2000, available from Machine Solutions Inc., Flagstaff, Ariz. Using the IDTE, the guide wire was advanced through each of the leads and into a test block and guide wire holding fixture until only 20±1 centimeters of the guide wire extended beyond a terminal pin. The proximal end of the guide wire/lead assembly was then extended proximal to a single roller assembly and statically clamped. The guide wire was clamped to minimize the natural bend in the lead but not put additional tension on the system or pull the guide wire out of the test model. The lead was then advanced through the 8Fr catheter and a representative tortuous anatomy path while monitoring the forces.

### Example 1

### Surface Roughness

To determine if an increase in surface roughness would decrease lead insertion and withdrawal forces between a lead and an 8Fr catheter when used in direct delivery cases, four groups of leads were formed having distinct surface finishes. To form the leads, a mold was first machined such that each of the mold cavities had a machined, smooth surface finish. The mold cavities were then roughened either by bead blasting or wire electric discharge machine burnishing.

SILASTIC BioMedical Grade Liquid Silicone Rubber Q7-4860 was then injected into the roughened mold. Because the silicone took the shape of the mold, it also naturally took on the roughness created during the molding process. The silicone was then allowed to cure or solidify in the mold. The surface roughness of the surfaces was measured using a NewView 6300 surface profiler, available from Zygo Corporation, Middlefield, Conn.

The first group of leads (L1) included the smooth machined surfaces prior to roughening and had an average surface roughness (Ra) of about 7 micro-inches. The second group of leads (L2) was roughened using wire electric discharge machine burnishing to an average surface roughness (Ra) of about 188 micro-inches. The third group of leads (L3) was roughed using a 70 Ra bead blast to an average surface roughness (Ra) of about 91 micro-inches. The fourth group of leads (L4) was roughed using a 100-FRa bead blast to an average surface roughness (Ra) of about 258 micro-inches. Due to the way the molds were made, the diameters of the leads varied slightly.

Table 1 shows the outer diameter and average surface roughness (Ra) of each of the leads and the peak insertion force and the peak withdrawal force of each of the leads. A force of less than about 380 grams-force was considered to be optimal.

TABLE 1

| | | Resultant | Peak | |
|---|---|---|---|---|
| | OD | Average Ra | Insertion Force | Peak Withdrawal Force |
| | (in) | (micro-inches) | (grams-force) | (grams-force) |
| L1 | 0.071 | 7 | 383.9 | 102.9 |
| L2 | 0.072 | 188 | 130.2 | 104.3 |
| L3 | 0.075 | 91 | 126.7 | 92.2 |
| L4 | 0.076 | 258 | 160.6 | 67.4 |

As illustrated by the results in Table 1, when the surface roughness was increased, the peak insertion forces of the leads in groups L2, L3 and L4 decreased compared to the peak insertion force of the leads in group L1. The leads having surface roughnesses of greater than about 91 Ra, resulted in acceptable force requirements.

### Example 2

### Surface Lubricity

To determine if an increase in the lubricity of a lead decreases lead insertion and withdrawal forces for an 8Fr catheter when used in direct delivery cases, two groups of leads were formed having different lubricious surfaces. The leads used in the first group (L5) were formed using DOW Q7-4860 and coated with LSR1-9716-30. The coating was applied using a manual dip coat process and cured at 100 degrees Celsius for 4 hours. The leads used in the second group (L6) were formed using MED1-4855 and did not include a coating.

Table 2 shows the number of samples tested (n), the peak insertion force and the peak withdrawal force of each of the leads. A force of less than about 227 grams-force was considered acceptable.

TABLE 2

| | | Peak Insertion Force | Peak Withdrawal |
|---|---|---|---|
| | n | (grams-force) | Force (grams-force) |
| L5 | 5 | 186.26 | 103.18 |
| L6 | 3 | 80.59 | 67.7 |

As can be seen by the data in Table 2, the leads in groups L5 and L6 had lead insertion forces that were within acceptable levels.

### Example 3

### Relationship between Maximum Insertion Force, Average Surface Roughness (Ra) and Peak to Valley Distance (PV)

The maximum insertion force was evaluated as a function of the average surface roughness (Ra) and peak to valley distance (PV) for several sample lead bodies.

To form the sample lead bodies, a mold was first machined such that each of the mold cavities had a machined, smooth surface finish. The mold cavities were then roughened either by bead blasting or wire electric discharge machine burnishing.

MED 1-4860 silicone rubber, a silicone material available from Nusil Silicone Technology LCC, Carpinteria, Calif., was then injected into the roughened mold. Because the silicone took the shape of the mold, it also naturally took on the roughness created during the molding process. The silicone was then allowed to cure or solidify in the mold. The average surface roughness and the peak to valley distance for each sample were measured using a NewView 6300 surface profiler, available from Zygo Corporation, Middlefield, Conn.

To determine the compatibility between various leads and a RAPIDO Advance catheter, the forces between each of the leads and the catheter were measured as each lead was pushed over a guide wire inserted into a test apparatus. The forces are representative of the forces a physician would experience upon introducing the lead through the 8Fr catheter placed in a worst case direct delivery position during delivery of the lead into a body.

The forces were measured on an Interventional Device Test Equipment (IDTE) 2000, available from Machine Solutions Inc., Flagstaff, Ariz. The leads were evaluated in water at 37° C. using a insertion rate of 20 inches/min. The smallest bend radius used to evaluate the sample leads was 0.5 inches. Using the IDTE, a guide wire was advanced through each of the leads and into a test block and guide wire holding fixture until only 20±1 centimeters of the guide wire extended beyond a terminal pin. The proximal end of the guide wire/lead assembly was then extended proximal to a single roller assembly and statically clamped. The guide wire was clamped to minimize the natural bend in the lead but not put additional tension on the system or pull the guide wire out of the test model. The lead was then inserted over the guide wire and through the catheter until the lead tip emerged from the catheter by at least 7 cm. The lead tip was then withdrawn from the catheter until the tip was in a straight portion of the catheter shaft (outside of the tortuous curves). Force versus displacement was recorded throughout the test.

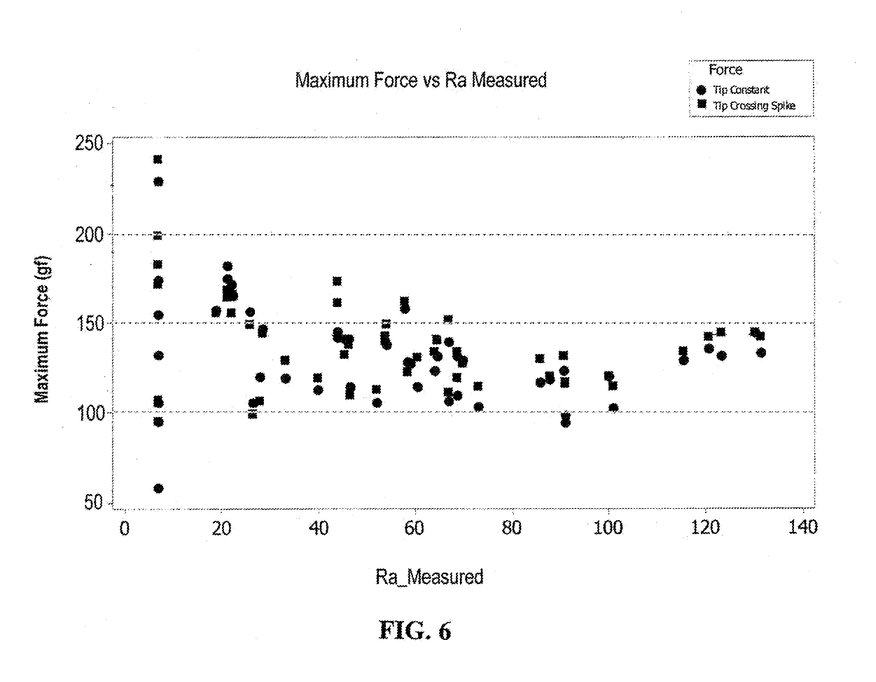

FIG. 6 is a scatter plot of the maximum insertion force versus the average surface roughness (Ra) for each of the sample lead bodies evaluated using the testing equipment described above. Each sample lead body evaluated generated two maximum insertion force values. The first value is representative of the “tip spike” value or rather the amount of force that is required to push the lead tip through the tip of the catheter when the catheter is placed in the tortuous anatomy. The second value is representative of the “tip constant” value. The tip constant value is representative of the maximum insertion force generated as the lead tip navigates the tortuous pathway of the test apparatus. The data appears below in Table 3.

TABLE 3

| | | Maximum Insertion |
|---|---|---|
| Sample No. | Ra (micro-inches) | Force (grams-force) |
| 1-1 | 6.8 | 198.95 |
| 1-2 | 6.8 | 173.74 |
| 2-1 | 6.8 | 183.21 |
| 2-2 | 6.8 | 155.09 |
| 3-1 | 6.8 | 106.20 |
| 3-2 | 6.8 | 95.13 |
| 4-1 | 6.8 | 107.74 |
| 4-2 | 6.8 | 105.81 |
| 5-1 | 6.8 | 95.13 |
| 5-2 | 6.8 | 58.07 |
| 6-1 | 6.8 | 172.05 |
| 6-2 | 6.8 | 132.67 |
| 7-1 | 6.8 | 241.55 |
| 7-2 | 6.8 | 229.87 |
| 8-1 | 18.7 | 155.57 |
| 8-2 | 18.7 | 157.22 |
| 9-1 | 21.0 | 164.85 |
| 9-2 | 21.0 | 174.90 |
| 10-1 | 21.0 | 168.37 |
| 10-2 | 21.0 | 181.95 |
| 11-1 | 22.0 | 155.77 |
| 11-2 | 22.0 | 171.51 |
| 12-1 | 22.3 | 166.30 |
| 12-2 | 22.3 | 164.99 |
| 13-1 | 25.7 | 149.39 |
| 13-2 | 25.7 | 156.73 |
| 14-1 | 26.3 | 99.48 |
| 14-2 | 26.3 | 105.66 |
| 15-1 | 27.7 | 106.92 |
| 15-2 | 27.7 | 120.50 |
| 16-1 | 28.3 | 144.17 |
| 16-2 | 28.3 | 146.39 |
| 17-1 | 33.0 | 128.71 |
| 17-2 | 33.0 | 119.77 |
| 18-1 | 39.7 | 119.53 |
| 18-2 | 39.7 | 112.96 |
| 19-1 | 43.7 | 161.27 |
| 19-2 | 43.7 | 145.43 |
| 20-1 | 45.3 | 132.72 |
| 20-2 | 45.3 | 140.84 |
| 21-1 | 46.0 | 138.13 |
| 21-2 | 46.0 | 141.13 |
| 22-1 | 46.3 | 109.58 |
| 22-2 | 46.3 | 114.89 |
| 23-1 | 52.0 | 112.86 |
| 23-2 | 52.0 | 105.95 |
| 24-1 | 53.7 | 142.67 |
| 24-2 | 53.7 | 139.48 |
| 25-1 | 54.0 | 149.05 |
| 25-2 | 54.0 | 137.60 |
| 26-1 | 57.7 | 161.95 |
| 26-2 | 57.7 | 158.08 |
| 27-1 | 58.3 | 122.67 |
| 27-2 | 58.3 | 128.32 |
| 28-1 | 59.0 | 127.21 |
| 28-2 | 59.0 | 127.26 |
| 29-1 | 60.3 | 130.55 |
| 29-2 | 60.3 | 114.46 |
| 30-1 | 64.0 | 133.69 |
| 30-2 | 64.0 | 123.15 |
| 31-1 | 64.3 | 140.31 |
| 31-2 | 64.3 | 131.32 |
| 32-1 | 66.7 | 111.08 |
| 32-1 | 66.7 | 106.44 |
| 33-1 | 66.7 | 151.27 |
| 33-2 | 66.7 | 139.24 |
| 34-1 | 68.7 | 119.53 |
| 34-2 | 68.7 | 109.53 |
| 35-1 | 68.7 | 133.59 |
| 35-2 | 68.7 | 131.27 |
| 36-1 | 69.7 | 127.12 |
| 36-2 | 69.7 | 128.95 |
| 37-1 | 73.0 | 114.46 |
| 37-2 | 73.0 | 103.59 |
| 38-1 | 85.7 | 129.68 |
| 38-2 | 85.7 | 116.68 |
| 39-1 | 87.7 | 120.50 |
| 39-2 | 87.7 | 118.47 |
| 40-1 | 90.7 | 131.13 |
| 40-2 | 90.7 | 123.73 |
| 41-1 | 100.0 | 120.06 |
| 41-2 | 100.0 | 119.97 |
| 42-1 | 101.0 | 114.36 |
| 42-2 | 101.0 | 102.52 |
| 43-1 | 115.3 | 133.54 |
| 43-2 | 115.3 | 128.90 |
| 44-1 | 120.7 | 142.05 |
| 45-2 | 120.7 | 135.91 |
| 46-1 | 123.3 | 144.27 |
| 47-2 | 123.3 | 131.46 |
| 48-1 | 130.3 | 144.65 |
| 48-2 | 130.3 | 144.22 |
| 49-1 | 131.3 | 141.61 |
| 49-2 | 131.3 | 132.82 |

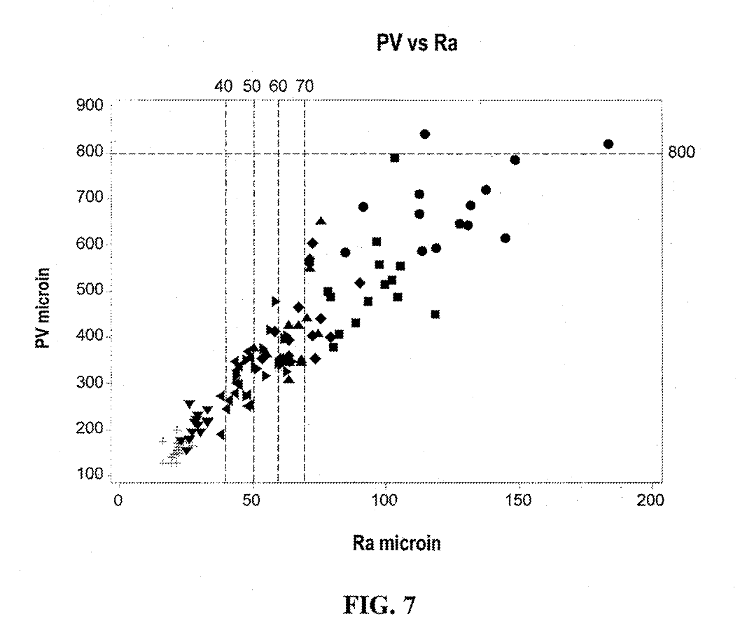

The relationship between the average surface roughness (Ra) and the maximum peak to valley distance (PV) was also evaluated. The maximum peak to valley distance (PV) for the sample lead bodies is plotted as a function of the average surface roughness (Ra) in FIG. 7 and the data appears below in Table 4.

TABLE 4

| | | Maximum PV |
|---|---|---|
| Sample No. | Ra (micro-inches) | (micro-inches) |
| 1-1 | 6.8 | — |
| 1-2 | 6.8 | — |
| 2-1 | 6.8 | — |
| 2-2 | 6.8 | — |
| 3-1 | 6.8 | — |
| 3-2 | 6.8 | — |
| 4-1 | 6.8 | — |
| 4-2 | 6.8 | — |
| 5-1 | 6.8 | — |
| 5-2 | 6.8 | — |
| 6-1 | 6.8 | — |
| 6-2 | 6.8 | — |
| 7-1 | 6.8 | — |
| 7-2 | 6.8 | — |
| 8-1 | 18.7 | 197.0 |
| 8-2 | 18.7 | 197.0 |
| 9-1 | 21.0 | 161.0 |
| 9-2 | 21.0 | 161.0 |
| 10-1 | 21.0 | 163.0 |
| 10-2 | 21.0 | 163.0 |
| 11-1 | 22.0 | 174.0 |
| 11-2 | 22.0 | 174.0 |
| 12-1 | 22.3 | 175.0 |
| 12-2 | 22.3 | 175.0 |
| 13-1 | 25.7 | 257.0 |
| 13-2 | 25.7 | 257.0 |
| 14-1 | 26.3 | 196.0 |
| 14-2 | 26.3 | 196.0 |
| 15-1 | 27.7 | 233.0 |
| 15-2 | 27.7 | 233.0 |
| 16-1 | 28.3 | 258.0 |
| 16-2 | 28.3 | 258.0 |
| 17-1 | 33.0 | 244.0 |
| 17-2 | 33.0 | 244.0 |
| 18-1 | 39.7 | 263.0 |
| 18-2 | 39.7 | 263.0 |
| 19-1 | 43.7 | 355.0 |
| 19-2 | 43.7 | 355.0 |
| 20-1 | 45.3 | 348.0 |
| 20-2 | 45.3 | 348.0 |
| 21-1 | 46.0 | 337.0 |
| 21-2 | 46.0 | 337.0 |
| 22-1 | 46.3 | 369.0 |
| 22-2 | 46.3 | 369.0 |
| 23-1 | 52.0 | 345.0 |
| 23-2 | 52.0 | 345.0 |
| 24-1 | 53.7 | 477.0 |
| 24-2 | 53.7 | 477.0 |
| 25-1 | 54.0 | 349.0 |
| 25-2 | 54.0 | 349.0 |
| 26-1 | 57.7 | 402.0 |
| 26-2 | 57.7 | 402.0 |
| 27-1 | 58.3 | 375.0 |
| 27-2 | 58.3 | 375.0 |
| 28-1 | 59.0 | 360.0 |
| 28-2 | 59.0 | 360.0 |
| 29-1 | 60.3 | 425.0 |
| 29-2 | 60.3 | 425.0 |
| 30-1 | 64.0 | 406.0 |
| 30-2 | 64.0 | 406.0 |
| 31-1 | 64.3 | 441.0 |
| 31-2 | 64.3 | 441.0 |
| 32-1 | 66.7 | 464.0 |
| 32-1 | 66.7 | 464.0 |
| 33-1 | 66.7 | 549.0 |
| 33-2 | 66.7 | 549.0 |
| 34-1 | 68.7 | 571.0 |
| 34-2 | 68.7 | 571.0 |
| 35-1 | 68.7 | 650.0 |
| 35-2 | 68.7 | 650.0 |
| 36-1 | 69.7 | 604.0 |
| 36-2 | 69.7 | 604.0 |
| 37-1 | 73.0 | 518.0 |
| 37-2 | 73.0 | 518.0 |
| 38-1 | 85.7 | 558.0 |
| 38-2 | 85.7 | 558.0 |
| 39-1 | 87.7 | 563.0 |
| 39-2 | 87.7 | 563.0 |
| 40-1 | 90.7 | 515.0 |
| 40-2 | 90.7 | 515.0 |
| 41-1 | 100.0 | 790.0 |
| 41-2 | 100.0 | 790.0 |
| 42-1 | 101.0 | 607.0 |
| 42-2 | 101.0 | 607.0 |
| 43-1 | 115.3 | 723.0 |
| 43-2 | 115.3 | 723.0 |
| 44-1 | 120.7 | 712.0 |
| 45-2 | 120.7 | 712.0 |
| 46-1 | 123.3 | 842.0 |
| 47-2 | 123.3 | 842.0 |
| 48-1 | 130.3 | 786.0 |
| 48-2 | 130.3 | 786.0 |
| 49-1 | 131.3 | 822.0 |
| 49-2 | 131.3 | 822.0 |

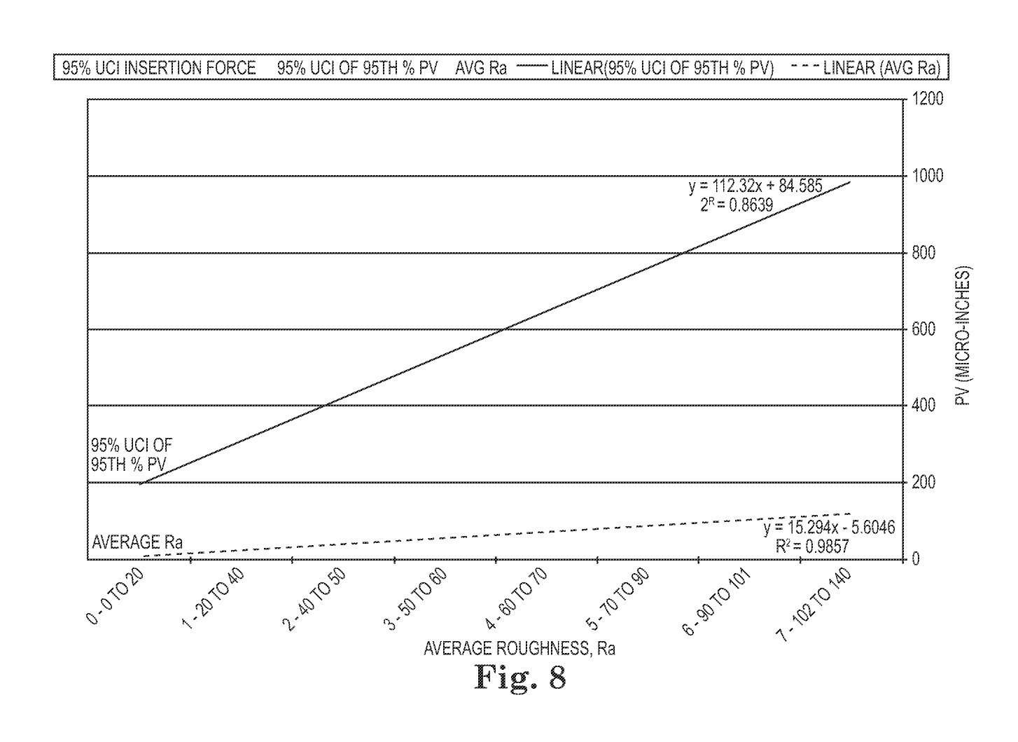

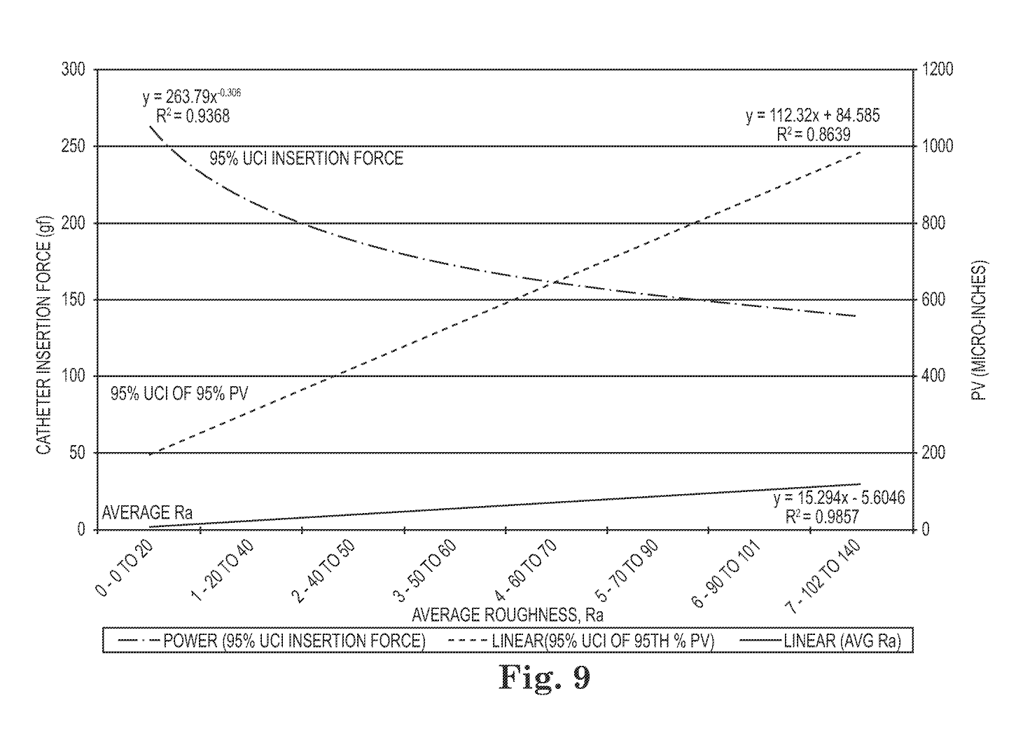

Next, using Microsoft EXCEL the linear relationship between the average surface roughness (Ra) and the maximum peak to valley distance (PV) was determined at the 95% upper confidence boundary for the 95% percentile of the range of Ra values. The graph is shown in FIG. 8.

Again, using Microsoft EXCEL, the relationship between average surface roughness (Ra) and the maximum insertion force at the 95% upper confidence boundary for the 95% percentile of the range of Ra values was evaluated. The graph is shown in FIG. 9. Also, plotted on the graph in FIG. 9 is the relationship between average surface roughness (Ra) and peak to valley distance (PV). As shown in FIG. 9, below a maximum insertion force below about 380 grams-force, the average surface roughness ranges from about 25 micro-inches to about 140 micro-inches and the maximum peak to valley distance ranges from about 200 micro-inches to about 800 micro-inches. Also, as shown in FIG. 9, in order to maintain a maximum insertion force below about 250 grams-force, the average surface roughness (Ra) ranges from about 40 micro-inches to about 90 micro-inches and the peak to valley distance (PV) ranges from about 200 micro-inches to about 650 micro-inches.

### Example 4

### Relationship between Maximum Insertion Force, Average Surface Roughness (Ra) and Catheter Clearance

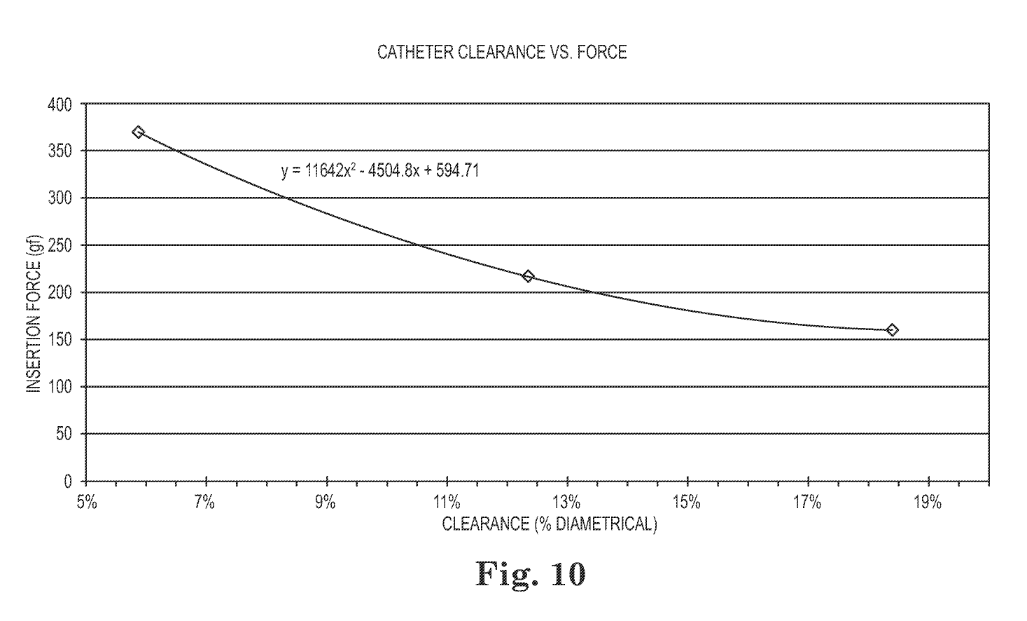

The maximum insertion force was evaluated as a function of catheter clearance for a group of sample lead bodies of differing outer diameters. The outer surfaces of the group of sample lead bodies were roughened such that they had the same average surface roughness of 70 micro-inches. Here, catheter clearance is defined as the percent difference between the inner diameter of the delivery catheter and the outer diameter of the lead body, (i.e. the sample lead body) including the roughened outer surface. The wall thickness of the catheters used in this evaluation was 0.008 inches. FIG. 10 is a graph showing the relationship between the maximum insertion force and catheter clearance. The data is presented in Table 5 below.

TABLE 5

| | % | Max. Insertion Force |
|---|---|---|
| Sample | Clearance | (grams-force) |
| 1 | 6 | 370 |
| 2 | 12 | 216 |
| 3 | 18 | 160 |

As shown in FIG. 10, the maximum insertion force generally decreases with an increase in the catheter clearance. To maintain a maximum insertion force of less than about 380 grams-force, the clearance between the catheter and the lead body should be at least 6% at an average surface roughness of 70 micro-inches. To maintain a maximum insertion force of less than about 250 grams-force, the amount of clearance between the catheter and the lead body should be at least about 10.5% at an average surface roughness of 70 micro-inches. Finally, to maintain a maximum insertion force of less than about 200 grams-force, the amount of clearance should be at least 14.5% at an average surface roughness of 70 micro-inches. As demonstrated in the above examples, the average surface roughness may also affect the amount of clearance needed between a select catheter and a lead body to remain under a target maximum insertion force threshold.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A method of forming a lead body for a medical electrical lead having a roughened outer surface, the method comprising:
roughening at least a portion of an inner surface of a mold cavity to an average surface roughness (Ra) of at least 7 micro-inches;
injecting a polymeric material into the roughened mold cavity for forming the lead body; and
curing the polymeric material to form a lead body having an outer surface wherein at least a portion of the outer surface is roughened to an average surface roughness Ra of at least 7 micro-inches.

2. The method of claim 1, wherein a maximum peak to valley distance is less than about 800 micro-inches.

3. The method of claim 1, further comprising roughening the inner surface of the mold cavity to an average surface roughness ranging from about 25 micro-inches to about 90 micro-inches and having a maximum peak to valley distance of less than about 800 micro-inches.

4. The method of claim 1, further comprising roughening the inner surface of the mold cavity to an average surface roughness ranging from about 7 micro-inches to about 65 micro-inches and having a maximum peak to valley distance of less than about 800 micro-inches.

5. The method of claim 1, further comprising roughening the inner surface of the mold cavity to an average surface roughness ranging from about 7 micro-inches to about 65 micro-inches and having a maximum peak to valley distance of less than about 650 micro-inches.

6. The method of claim 1, further comprising roughening the inner surface of the mold cavity to an average surface roughness ranging from about 40 micro-inches to about 65 micro-inches and having a maximum peak to valley distance of less than about 650 micro-inches.

7. The method of claim 1, wherein the polymeric material comprises silicone.

8. The method of claim 1, wherein roughening at least a portion of the mold cavity comprises any one of bead blasting the mold, wire electric discharge machine burnishing the mold or chemical etching the mold cavity.

9. The method of claim 1, wherein silicone rubber is injected into the mold cavity to form a distal tip region of a lead body having a roughened outer surface.

10. A method of roughening an outer surface of a least a portion of a lead body for a medical electrical lead, the method comprising:
machining a mold including a cavity for forming a lead body such that an inner surface of the cavity is substantially smooth;
wire electric discharge machine burnishing the inner surface of the cavity to roughen at least a portion of the inner surface of the cavity to an average surface roughness (Ra) ranging from about 7 micro-inches to about 90 micro-inches and having a maximum peak to valley distance of less than about 800 micro-inches;
injecting a polymeric material comprising silicone into the roughened cavity for forming the lead body; and
curing the polymeric material to form a lead body having an outer surface wherein at least a portion of the outer surface is roughened to an average surface roughness (Ra) ranging from about 7 micro-inches to about 90 micro-inches and having a maximum peak to valley distance of less than about 800 micro-inches.

11. The method of claim 10, further comprising roughening the inner surface of the cavity to an average surface roughness ranging from about 25 micro-inches to about 90 micro-inches.

12. The method of claim 10, further comprising roughening the inner surface of the cavity to an average surface roughness ranging from about 7 micro-inches to about 65 micro-inches and having a maximum peak to valley distance of less than about 650 micro-inches.

13. The method of claim 10, further comprising roughening the inner surface of the cavity to an average surface roughness ranging from about 40 micro-inches to about 65 micro-inches and having a maximum peak to valley distance of less than about 650 micro-inches.

14. The method of claim 10, wherein silicone rubber is injected into the cavity to form a distal tip region of lead body.

15. A medical electrical lead having a reduced contact area, the lead comprising:
an elongated polymeric lead body extending from a proximal end adapted to be coupled to a pulse generator to a distal end, wherein at least a portion of the elongated polymeric lead body has a roughened outer surface having an average surface roughness ranging from about 25 micro-inches to about 90 micro-inches and having a maximum peak to valley distance of less than about 800 micro-inches;
at least one conductor extending within the lead body from the proximal end in a direction towards the distal end; and
at least one electrode located on the lead body and operatively coupled to the at least one conductor.

16. The medical electrical lead of claim 15, wherein the roughened outer surface has an average surface roughness ranging from about 40 micro-inches to about 90 micro-inches.

17. The medical electrical lead of claim 15, wherein the roughened outer surface has an average surface roughness ranging from about 7 micro-inches to about 90 micro-inches and having a maximum peak to valley distance of less than about 650 micro-inches.

18. The medical electrical lead of claim 15, wherein the roughened outer surface has an average surface roughness ranging from about 40 micro-inches to about 65 micro-inches and having a maximum peak to valley distance of less than about 650 micro-inches.

19. The medical electrical lead of claim 15, wherein the at least one portion of the lead body having the roughened outer surface comprises silicone.

20. The medical electrical lead of claim 15, wherein the at least one portion is a distal tip region of the lead body, wherein the distal tip region comprises silicone.
